(19) United States
(12) Patent Application Publication
(10) Pub. No.: US 2018/0078410 A1
(43) Pub. Date: Mar. 22, 2018

(54) Surgery Device

(71) Applicant: Cosmin-Adrian Gavanescu, Targu Jiu (RO)

(72) Inventor: Cosmin-Adrian Gavanescu, Targu Jiu (RO)

(21) Appl. No.: 15/385,882

(22) Filed: Dec. 21, 2016

Related U.S. Application Data
(60) Provisional application No. 62/396,888, filed on Sep. 20, 2016.

(51) Int. Cl. A61F 9/007; A61B 17/32; A61B 10/02

(52) U.S. Cl. CPC A61F 9/007; A61F 9/00736; A61B 2017/320064; A61B 10/0283; A61B 17/32

(57) ABSTRACT

The embodiments provide a device for eye surgery consisting of two metal tubes of cylindrical shape, arranged concentrically with respect to one another, the outer tube is fixed, and the inner tube is rotated by means of mechanical, electromechanical, or piezoelectric and/or pneumatic actuator, or by means of compressed air. The actuators are placed at the proximal end of the device and/or at the base of the device and/or in a compartment separated from it at a short distance from the tip. The diameter of the outer tube gradually decreases, forming a region in the shape of a truncated cone, which ends with a flat or rounded end-piece.

### CROSS-REFERENCE TO RELATED APPLICATION

This Continuation-In-Part application claims priority to the pending Non-Provisional patent application (application Ser. No. 15/385,882” titled “Surgery Device” filed on Dec. 21, 2016, by Cosmin-Adrian Gavanescu.

### FIELD

The invention relates to a device for coagulation, cutting and aspiration, with the possibility of visualizing through a fiber optic visioning system, which can be used in microsurgery (eye surgery, neurosurgery, plastic surgery, ENT, and/or other surgical fields), in laparoscopic surgery, endoscopic surgery, general surgery-, gynaecology, orthopedic and/or other human and/or animal surgical fields requiring more or less selective excision and/or cutting of normal and/or abnormal tissues (e.g. tumors, cysts, and/or other forms of physiological and/or pathological lesions). The dimensions of this device can be adapted both for use at microscopic level under conventional microscope, and/or endoscopically projecting an imagine on a screen, and/or to its use at macroscopic level, depending on the amount of space available for maneuvering and depending on the type of surgery' it needs to be used in.

### BACKGROUND

There is a known device with the disposable endo-coagulator laser used in ophthalmic surgery according to U.S. Pat. No. 4,537,193. device that includes an elongated hand tool provided with a stainless steel probe, positioned at the leading end of the tool, which has an optical fiber inserted into a hole of the tool, so that the fiber ends at the distal end of the probe, the optical fiber is attached to a connector comprising a plastic body with a metal plug introduced therein, metal plug made of stainless steel, worked with great precision so that the optical fiber can be accurately positioned and aligned with respect to the conventional argon-ion laser beam.

In addition, it is also known in KR20140103435 a surgical stapling instrument used in laparoscopic surgery, which is characterized by a rod-shaped bar which moves inside the abdominal cavity by trocar system, consisting of a hook part placed on the bar, to form an inlet for the suturing thread entering the inlet and allowing the suture and an opening and closing lid for the thread entering the inlet, which tool reduces the time to suture during laparoscopic surgery and the surgery can be performed easily.

U.S. Pat. App. No. 2012/041358 discloses a device to operate with one hand to irrigate and aspirate the eye which has a tube with a tip at a front end. The tip being an outlet of the irrigation fluid, and a suction opening for taking the material to be extracted by the suction; an irrigation fluid pump is disposed in the tube and it is connected to the output of the irrigation fluid, a suction pump being fitted in an irrigation pipe inside the tube, connected to the suction inlet through a suction pipe, and a controller regulates the flow through the irrigation pipe so as to be equal to the flow through the suction pipe, preferably the device be provided with a cutting/milling tool which has the possibility of rotating in the suction inlet. Devices that use a laser for destructing the tissue do not offer the possibility′ of aspirating the cauterized material. This makes the interventions for glaucoma to be an ineffective solution because the cauterized tissue remaining in the eye will inevitably lead to clogging of collector channels and ultimately to increased intra-ocular pressure. (U.S. Pat. No. 4,537,193). There is a device for intra-ocular surgery that uses laser cauterization and offers the possibility of vacuuming the burnt material, but the outer diameter of the device is too large to be used in the restricted space between the iris and the cornea.

The devices used in laparoscopic surgery perform cutting by clamping and severing, thus requiring more than one operation, while the proposed solution allows the high-precision cutting of the tissue. Since the device only cuts the tissue entering through the opening in the outer tube, it allows a very fine and precise control of the amount of material cut and the affected area. Suture devices do not provide the opportunity to cut-the-material and remove it by suction (K.R20140103435)

The devices using a high-intensity electric field such as disclosed in U.S. Pat. No 2011/144638A1 for destroying the tissue only achieves a temporary separation of the proteins in the tissue, which is not sufficient to detach the trabecular meshwork: on the contrary, it may result in a repositioning of the tissue which will further prevent the removal of the intra-ocular fluids. Such a process cannot cut tissues with harder consistency such as the trabecular meshwork.

The problem to be solved by the present invention consists in the possibility of coagulation, removal, and suction of different types of tissues of the eye and/or from various parts of the human body, with the possibility of visualizing through a fiber optic visioning system that can project images on a screen.

This proposed solution to the illustrated issue is achieved by a device for eye surgery consisting of two metal tubes of cylindrical shape, arranged concentrically with respect to one another, the outer tube is fixed, and the inner tube is rotated by means of mechanical, electromechanical, or piezoelectric and/or pneumatic actuator, or by means of compressed air. The actuators are placed at the proximal end of the device and/or at the base of the device and/or in a compartment separated from it at a short distance from the tip. The diameter of the outer tube gradually decreases, forming a region in the shape of a truncated cone, which ends with a flat or rounded end-piece. Near the top of the device, the outer tube has an opening, which overlaps with one or more openings in the inner tube, symmetrically placed on the surface of this inner truncated cone, and which, by rotation of the inner tube, the inner opening(s) slides under the opening of the outer tube, thus cutting the tissue/material, which is sucked and discharged to the base of the device due to a negative pressure created inside the inner tube.

### SUMMARY OF THE INVENTION

The surgery device consists of two metal tubes of cylindrical shape, one outer tube (1) and one inner tube (2) that are arranged concentrically with respect to one another. The outer tube (1) being fixed while the inner tube (2) can be rotated around its longitudinal axis by means of mechanical, electromechanical, and/or piezoelectric actuators, and/or by means of pneumatic (air or another) actuators. The actuators can be placed at the proximal part and/or at the base of the device and/or in a compartment separated from it at a short distance from the tip. The outer tube can be enveloped with fiber optic filaments that can be used for illumination, imaging, and laser coagulation. The irrigation/inflation system consists of a cylindrically shaped hollow piece that slides onto the outer tube and on top of the finer optic envelope. The piece has an opening that is placed on the opposite side with respect to the cutting opening. Through this opening fluid (liquid or gas) is pumped in order to keep the pressure inside the virtual cavity (eye, peritoneum, and/or others) at a desired value. The flow of fluid is controlled by a system, not illustrated here, that is placed at some distance away from the end piece. This system maintains the desired pressure inside the virtual cavity where the device works in, by controlling both the amount of suction from the inner tribe and the irrigation from this outer sleeve. The tubes (1) and (2) are forming at the distal part an area in the shape of a truncated cone which ends with an end-piece (3), and near the tip of the device, the outer tube (1) has an opening (4), which may overlap with one or more openings (5) of the inner tube (2), symmetrically placed on the surface of the inner truncated cone, and which, by rotation of the inner tube, they are sliding under the opening (4) of the outer tube (1), thus cutting the tissue/material, which is sucked and discharged to the base of the device due to a negative pressure created inside the inner tube (2) The fiber optic envelope contains fibers responsible for illumination, fibers that will serve to have a light source inside the eye and/or other organ and/or body, fibers that will be responsible to capture the image and carried to a visioning system that will recreate a bi-dimensional and/or tridimensional imagines that can be projected on a screen and the third type of fibers will be responsible for the transmission of the laser radiation used for photocoagulation. This fiber optic envelope can be placed on different others intra-ocular instruments (scissors, forceps, picks, hooks, and/or others) and/or other instruments used in other human surgical fields, and in combination with images capture by a second instrument and/or device having the same envelope, with the help of a visioning system with an integrating video software, not illustrated here, to measure and recreate a tridimensional image that can be projected on a screen.

Other aspects, advantages, and novel features of the embodiments shown herein will become apparent from the following detailed description in conjunction with the drawings.

### BRIEF DESCRIPTION OF THE DRAWINGS

A more complete understanding of the embodiments shown, and the attendant advantages and features thereof, will be more readily understood by reference to the following detailed descriptions when considered in conjunction with the accompanying drawings wherein:

FIG. 1 is a cross-sectional view of an advanced surface ablation apparatus;

FIG. 2 is a perspective view of the exterior tube of the apparatus;

FIG. 3 is a perspective view of the interior tube of the apparatus; and

FIG. 4 is a view of a fiber optic image of the apparatus in use.

### DETAILED DESCRIPTION

The specific details of a single embodiment or variety of embodiments described herein are set forth in this application. Any specific details of the embodiments are used for demonstration purposes only and no unnecessary limitations or inferences are to be understood therefrom.

The embodiments relate to a device for coagulation, cutting and aspiration, with the possibility of visualizing through a fiber optic visioning system, which can be used in microsurgery (eye surgery, neurosurgery, plastic surgery, ENT, and/or other surgical fields), in laparoscopic surgery, endoscopic surgery, general surgery-, gynaecology, orthopedic and/or other human and/or animal surgical fields requiring more or less selective excision and/or cutting of normal and/or abnormal tissues (e.g. tumors, cysts, and/or other forms of physiological and/or pathological lesions). The dimensions of this device can be adapted both for use at microscopic level under conventional microscope, and/or endoscopically projecting an imagine on a screen, and/or to its use at macroscopic level, depending on the amount of space available for maneuvering and depending on the type of surgery' it needs to be used in.

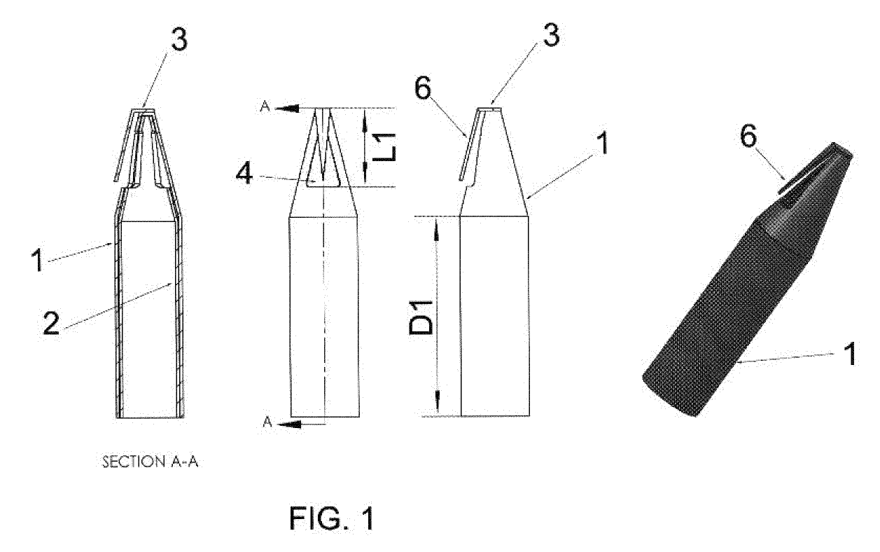

Referring now to the drawings wherein like reference numerals designate identical or corresponding parts throughout the views. There is shown in FIG. 1 a device for eye surgery, microsurgery (eye surgery, glaucoma surgery, vitreoretinal surgery, neurosurgery, plastic surgery, ENT, and/or other surgical fields), in laparoscopic surgery, general surgery', gynecology, orthopedic and/or other human and/or animal surgical fields characterized by that it consists of two metal tubes of cylindrical shape, having a tip in the shape of a truncated cone, an outer tube (1) and an inner tube (2), arranged concentrically with respect to one another, the outer tube (1) being fixed and the inner tube (2) being rotated by means of mechanical, electromechanical or piezoelectric actuator, or by means of compressed air, the actuators being placed at the base of the device and/or in a compartment separated from it; the diameter of the outer tube gradually decreases near the tip, forming a region in the shape of a truncated cone, which ends or not with an end-piece (3), and near the top of the device, the outer tube has an opening (4), which may overlap with some openings (5) in the inner tube (2). symmetrically placed on the surface of the truncated cone, and which, by rotation of the inner tube, slides under the opening (4) of the outer tube (1), thus cutting the material, which is sucked and discharged to the base of the device due to a negative pressure created inside the inner tube (2).

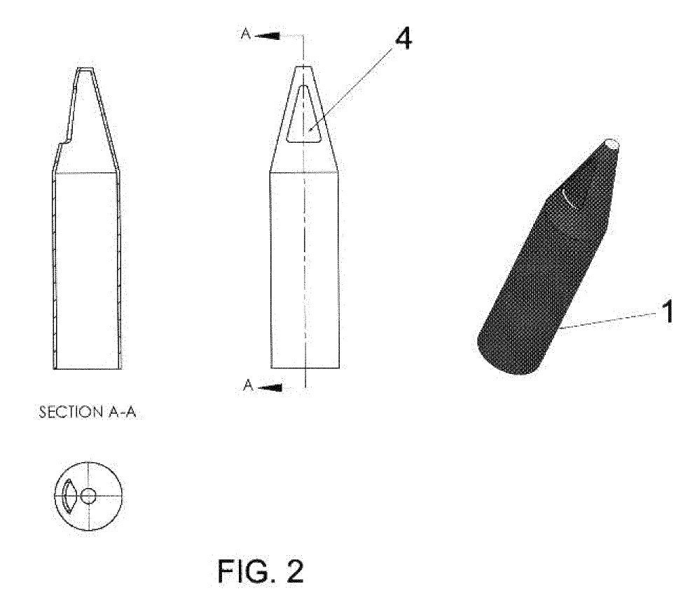

Shown in FIG. 2 is the device for eye surgery according to the invention consists of two metal tubes 1 and 2 that are arranged concentrically with respect to one another. The outer tube 1 is fixed, while the inner tube 2 is set in motion by means of mechanical electromechanical and/or piezoelectric, and/or pneumatic (air or another) actuators, or by means of compressed air. The actuators are placed at the proximal part, or are placed at the base of the device and/or in a compartment separated from it.

The two tubes 1, 2 are made of steel, titanium, carbon fiber, polycarbonate, plastics, ceramic material, and/or any other materials that are compatible with the scope of the invention. The tubes 1, 2 have a cylindrical shape, and the tip can be cut and/or formed into various shapes. At a short distance from the tip, the diameter D1 of the outer tube 1 decreases gradually, forming a region in the shape of a truncated cone, which ends with an end piece 3 of the same material. The end piece 3 may also be made of another material which is able to maintain sealed the outer tube 1.

The outer tube 1 has an opening 4 which may have a length L1 up to half the diameter of the tube 1 and whose size is different depending on the diameter of the outer tube 1. The opening 4 is positioned near the tip of the device, on the part of the area shaped like a truncated cone. The distance from the beginning of the opening 4 to the tip may vary depending on the diameter of the tube 1.

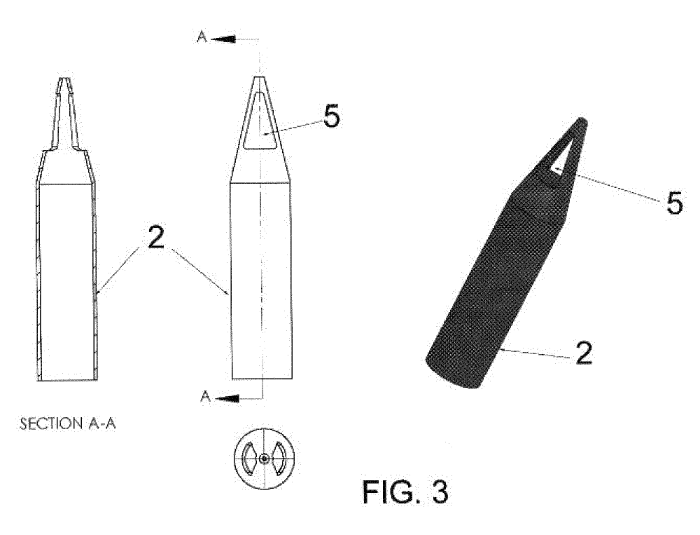

The inner tube 2 has a shape similar to that of the outer tube 1, but has a smaller diameter than the diameter of the outer tube 1 so that it can slide into it. In the vicinity of the tip, this inner tube 2 also has an area in the shape of a truncated cone, with an opening 5. as shown in FIG. 2, two openings 5 as shown in FIG. 3, or more openings, placed symmetrically on the surface of the tube.

By means of a kinematic chain and/or gears, not shown, which are connected to the inner tube 2, it rotates around its longitudinal axis inside the outer tube 1. In this way, the openings 4 and 5 slide with one another and carry out the cutting of the material.

Inside the inner tube, 2 low negative pressure is created, aspiring the sliced tissue/material by inserting it into the inner tube 2 and then evacuating it to the base of the device.

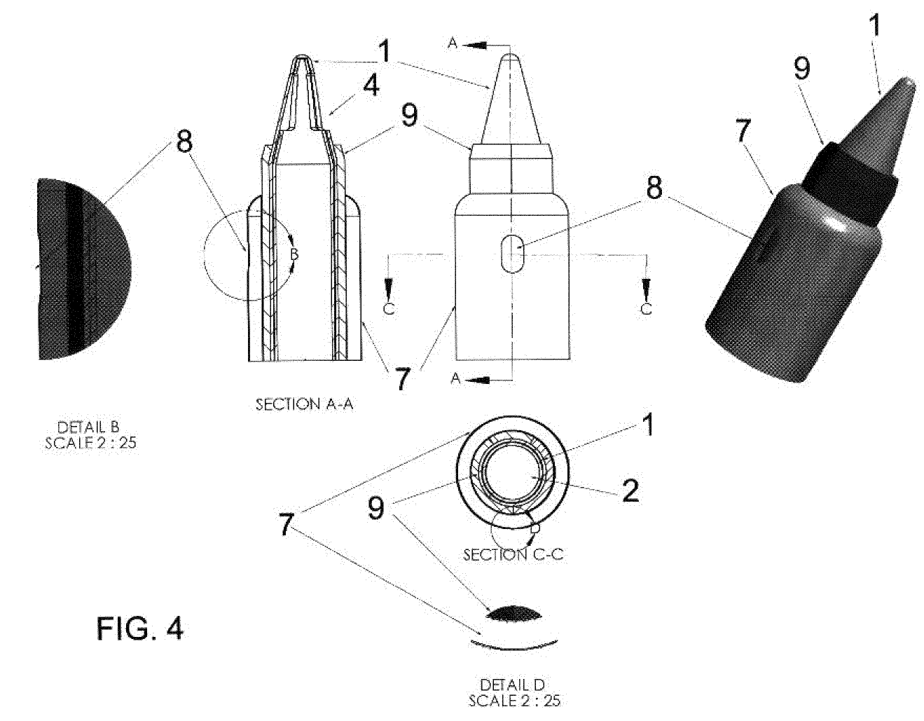

The variation of this device involves the integration of an irrigation system in addition to the cutting, suction, and fiber optic layer, all in one instrument as seen in FIG. 4. The irrigation/inflation system consists of a cylindrically shaped hollow piece 7 that slides onto the outer tube, and on top of the finer optic envelope 9. The piece has an opening 6 that is placed on the opposite side with respect to the cutting opening 4. Through this opening fluid (liquid or gas) is pumped in order to keep the pressure inside the virtual cavity (eye, peritoneum, etc.) at a desired value. The flow of fluid is controlled by a system, not illustrated here, that is placed at some distance away from the end piece. This system maintains the desired pressure inside the virtual cavity that the device works in, by controlling both the amount of suction from the inner tube and the irrigation from this outer sleeve.

The two tubes may have very similar diameters only in the area shaped like a truncated cone, and in the tubular area, the diameter of the inner tube 2 must be smaller so that there's a gap between the two tubes. Through the space between the two tubes, there may circulate a fluid (liquid or gas) carried out through an opening in the outer tube 1 other than 5. In this way the irrigation of the eye/organ/cavity can be achieved to maintain a nominal pressure in the desired range.

At the tip of the outer tube 1 there can be attached a sharp blade 6, which extends from the end point and, at a certain distance, it forms a curve off the tube, passes over the opening 4 and ends at the edge of the opening, covering the entire length of the opening or just a section thereof.

From the point of bending the blade 6 gradually narrows until it reaches the shape of a sharp tip.

The outer tube 1 has markings on its outer surface, as continuous lines around, spaced equally, and intermediate lines covering only half the diameter of the part.

The outer tube can be enveloped with fiber optic filaments 9 that can be used for illumination, imaging, and laser coagulation. Different types of fiber optics can be used depending on their utilization. Optic fibers can extend onto the truncated cone section, but not beyond the cutting opening 4.

In one variation, all three types of fibers are used such that the fibers form a cylindrically shaped piece that envelopes the outer tube. The fibers from the immediate vicinity of the cutting opening can be used to send images to a visioning system, not illustrated here, that can project images on a screen. The imaging fibers are isolated by means of light blocking material from the other fibers found on each side. Using the images captured from inside the cavity the visioning system can measure and recreate a bi-dimensional and/or tridimensional image that can be used to point the laser fibers with great precision towards the material to be irradiated, coagulated and/or cauterized. If any residual material is resulting from the laser exposure, it can be removed using the cutting opening of the instrument. The fiber optic envelope contains fibers responsible for illumination, fibers that will serve to have a light source inside the eye and/or other organ and/or body, fibers that will be responsible to capture the imagine and carried to a visioning system that will recreate a bi-dimensional and/or tridimensional imagine that can be projected on a screen and the third type of fibers will be responsible to carry the laser signal for photocoagulation.

Shown in FIG. 3 and FIG. 4 is a view of the device for surgery according to Claim 1 characterized by the fact that the outer tube (1) is enveloped with fiber optic filaments (9) that can be used for illumination, imaging and laser coagulation The fiber optic envelope contains fibers responsible for illumination, fibers that will serve to have a light source inside the eye and/or other organ and/or body, fibers that will be responsible to capture the image and carried to a visioning system that will recreate a bi-dimensional and/or tridimensional imagines that can be projected on a screen and the third type of fibers will be responsible to carry the laser signal for photocoagulation. This fiber optic envelope can be placed on different others intra-ocular instruments, and/or other instruments used in other human surgical fields, and in combination with images capture by a second instrument and/or device having the same envelope, with the help of a visioning system with an integrating video software, not illustrated here, to measure and recreate a tridimensional image that can be projected on a screen. The irrigation system consists of a cylindrically shaped hollow piece (7) that slides onto the outer tube and on top of the finer optic envelope (9). The piece has an opening (8) that is placed on the opposite side with respect to the cutting opening (4). Through this opening fluid is pumped in order to keep the pressure inside the eye at a desired value. The flow of fluid is controlled by a system, not illustrated here, that is placed at some distance from the end piece and that permits by controlling the suction from the inner tube and the irrigation to maintain the desired pressure in the cavity. This device can be used in glaucoma surgery with the irrigation system or without that in vitreoretinal surgery and/or other types of surgery fields.

It will be appreciated by persons skilled in the art that the present invention is not limited to what has been particularly shown and described hereinabove. In addition, unless mention was made above to the contrary, it should be noted that all of the accompanying drawings are not to scale. A variety of modifications and variations are possible in light of the above teachings without departing from the following claims.

What is claimed is:

1. A device for removing a tissue during a medical procedure and viewable through a fiber optic projection means, the device comprising:
a substantially cylindrical shaped and metallic outer port having a plurality of apertures about a cone-shaped first end and configured to store a removed tissue;
a dimensioned inner tube affixed within the metallic outer port and configured to cut a tissue by a pneumatic actuator;
a fiber optic visioning mechanism releasably attached about a diameter of the outer port to provide at least a two-dimensional image during a microsurgical procedure.

2. The device of claim 1, wherein the first end of the outer port further includes a tissue sensing portion.

3. The device of claim 1, wherein the outer port further includes a hollow inner cavity at a second end to store a removed tissue.

4. The device of claim 3, wherein the second end of the outer port further provide a coupling to releasably attached to at least one pneumatic hose.

5. The device of claim 1, wherein the dimensioned inner tube further includes a plurality of cutting blades affixed about a diameter of a cutting member at a first end.

6. The device of claim 5, wherein the dimensioned inner tube further enables a controlled negative pressure to discharge a removed tissue to the hollow inner cavity of the outer port.

7. The device of claim 1, wherein the fiber optic visioning mechanism further laser coagulation.

8. The device of claim 1, wherein further including an irrigation mechanism attached to a portion of the outer port and configured to provide a pre-determined fluid flow to the virtual cavity.

9. A microsurgical device for removing tissue, the device comprising:
a substantially cylindrical outer tube configured to house an inner cutting tube and further including an outward protruding cutting blade at a first end;
an inner cutting tube having a plurality of cutting blades and configured to rotate about an axis to cut and remove a selected tissue.
a pneumatic actuator releasably attachable to the outer tub and configured to move the inner cutting tube relative to the outer tube;
a fiber optic visioning mechanism releasably attached to the outer port and configured to transmit an image to at least a two-dimensional monitoring platform; and
an irrigation means releasably attached to a perimeter of the outer tube and configured to provide a fluid communication to a virtual cavity.

10. The device of claim 7, where the outer port further includes a first triangular-shaped aperture at the first end and configured to capture a selected tissue between the rotating inner cutting tube and outward protruding cutting blade.

11. The device of claim 8, wherein the outward protruding cutting blade is further dimensioned to correspond to the first triangular-shaped aperture.

12. The device of claim 8, wherein the outer port further includes a second triangular-shaped aperture opposite the first triangular shaped aperture and configured to provide a passageway for fluid flow between the irrigation means and the virtual cavity.

13. The device of claim 7, wherein the fiber optic visioning mechanism is further configured to provide a laser coagulation to the internal cavity.

14. The device of claim 8, wherein the fiber optic visioning mechanism further includes a plurality of illumination fibers to provide a light source to the internal cavity.

15. The device of claim 11, wherein the fiber optic visioning mechanism is further dimensioned to fit a plurality of intra-ocular instruments.

16. The device of claim 9, wherein the outer port further includes a hollow inner chamber at a second end to store the removed tissue capture in a negative vacuum created by the rotating inner cutting tube.

17. The device of claim 7, wherein the actuator further includes at least a piezoelectric actuator.

18. The device of claim 7, wherein the irrigation means further includes a regulator to control a desired pressure of the fluid flow to the virtual cavity.

19. A microsurgical device for removing tissue, the device comprising:
a substantially cylindrical outer tube configured to house an inner cutting tube and further including;
a plurality of apertures to provide an access to the inner cutting tube and provide an irrigation to an internal cavity:
a cutting blade releasably attached to a tip of the first end and dimensioned to cover at least one of the plurality of apertures of the cylindrical outer tube;
a hollow internal region to releasably store a removed tissue;
an inner cutting tube configured to rotate about an axis at a pre-determined speed and affect the removed tissue from the internal cavity;
an actuator configured to fit onto a second end of the cylindrical shaped outer tube and provide a pneumatic means to the inner cutting tube;
a fiber optic visioning mechanism releasably attached to the outer port and configured to:
transmit at least a two-dimensional image to a display unit;
enable laser photocoagulation;
selectively illuminate the internal cavity; and
an irrigation mechanism configured to be releasably attached about a perimeter of the cylindrical outer port and provide a fluid flow to the internal cavity.

20. The device of claim 19, wherein the inner cutting tube is further configured to create a negative vacuum while in use.
